(19) United States
(12) Patent Application Publication
(10) Pub. No.: US 2012/0110733 A1
(43) Pub. Date: May 10, 2012

(54) Flexible Pillow

(76) Inventor: William John Salvaggio, Brewster, MA (US)

(21) Appl. No.: 12/940,686

(22) Filed: Nov. 5, 2010

(51) Int. Cl. A47C 21/04; A47G 9/10; A61F 7/00

(52) U.S. Cl. 5/421; 5/652

(57) ABSTRACT

A flexible, elongated pillow with one or more openings for inserting heating devices, vibrating devices, additional cushioning or standard heating blankets. The pillow has a shape, length and flexibility to allow for use as a neck pillow, a shoulder pillow or a pillow for both feet when lying in a supine position.

### FIELD OF THE INVENTION

The field of the invention relates to a flexible elongated pillow with an opening for inserting heating devices, vibrating devices, additional cushioning or standard heating blankets. The pillow has a shape, length and flexibility to allow multiple uses, including neck pillow, shoulder pillow and a pillow for both feet when lying in a supine position.

### BACKGROUND OF INVENTION

Pillows have been designed to accommodate cushioning different body parts. These pillows are shaped to complement these different body parts to provide targeted cushioning. A user must acquire and store all these specifically designed pillows to accommodate all the different body parts. This is burdensome, expensive and inefficient for a person to achieve all the desired results.

Furthermore, a pillow specifically designed to provide cushioning and heat to both feet when a user is lying in the supine, or face upwards, position does not exist.

Accordingly, a pillow is needed that can provide cushioning to both feet of a user in the supine position, and can also be versatile to provide targeting cushioning to a user's shoulders, neck, arms, back and head.

### SUMMARY OF THE INVENTION

There are additional features of the invention that will be described hereinafter and which will form the subject matter of the claims appended hereto. In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of the description and should not be regarded as limiting.

The subject invention discloses a pillow comprising: a substantially tubular shaped body, wherein the body is composed of soft materials, further wherein the body comprises a first end, a second end and a central portion, wherein the central portion has a sufficient length and the body has sufficient flexibility for a user to wrap the central portion around neck of the user with the first end contacting the upper right chest of the user and the second end contacting the upper left chest of the user; a heating unit contained within the body, wherein the heating unit is operational coupled to a power supply; a cover configured for covering the external surface of the body; and a user actuator attached to the external surface of the body, wherein the actuator is operational coupled to the power supply and adapted to activate the heating unit.

Another embodiment of the subject invention is a pillow comprising: a substantially tubular shaped body, wherein the body is composed of soft materials, further wherein the body comprises a first end, a second end and a central portion, wherein the central portion has a sufficient length and the body has sufficient flexibility for a user to wrap the central portion around neck of the user with the first end contacting the upper right chest of the user and the second end contacting the upper left chest of the user; a first opening on the first end; a second opening on the second end; an internal channel within the body for slidably receiving an insert through either the first or second opening; and a cover configured for covering the external surface of the body and the internal surface of the internal channel.

A further embodiment of the subject invention is a pillow comprising: a substantially tubular shaped body, wherein the body is composed of soft materials, further wherein the body comprises a first end, a second end and a central portion, wherein the central portion has a sufficient length and the body has sufficient flexibility for a user to wrap the central portion around neck of the user with the first end contacting the upper right chest of the user and the second end contacting the upper left chest of the user; a first opening on the first end; a second opening on the second end; an internal channel within the body for slidably receiving an insert through either the first or second opening; a cover configured for covering the external surface of the body and the internal surface of the internal channel; a heating unit contained within the body, wherein the heating unit is operational coupled to a power supply; and a user actuator attached to the external surface of the body, wherein the actuator is operational coupled to the power supply and adapted to activate the heating unit.

An additional embodiment of the subject invention is a pillow comprising: a substantially tubular shaped body, wherein the body is composed of soft materials, further wherein the body comprises a first end, a second end and a central portion, wherein the central portion has a sufficient length and the body has sufficient flexibility for a user to wrap the central portion around neck of the user with the first end contacting the upper right chest of the user and the second end contacting the upper left chest of the user; a first opening on the first end; an internal channel within the body for slidably receiving an insert through the first opening; and a cover configured for covering the external surface of the body and the internal surface of the internal channel.

Another embodiment of the subject invention is a pillow comprising: a substantially tubular shaped body, wherein the body is composed of soft materials, further wherein the body comprises a first end, a second end and a central portion, wherein the central portion has a sufficient length and the body has sufficient flexibility for a user to wrap the central portion around a shoulder of the user with the first end contacting the second end underneath an arm of the user; a first opening on the first end; an internal channel within the body for slidably receiving an insert through the first opening; and a cover configured for covering the external surface of the body and the internal surface of the internal channel.

A further embodiment of the subject invention is a pillow comprising: a substantially tubular shaped body, wherein the body is composed of soft materials, further wherein the body comprises a first end, a second end and a central portion, wherein the central portion has a sufficient length to lay over both feet of a user lying in a supine position; a first opening on the first end; an internal channel within the body for slidably receiving an insert through the first opening; and a cover configured for covering the external surface of the body and the internal surface of the internal channel.

Another embodiment of the subject invention is a pillow comprising: a substantially tubular shaped body, wherein the body is composed of soft materials, further wherein the body comprises a first end, a second end and a central portion, wherein the central portion has a sufficient length to lay underneath both feet of a user lying in a supine position; a first opening on the first end; an internal channel within the body for slidably receiving an insert through the first opening; and a cover configured for covering the external surface of the body and the internal surface of the internal channel.

In other embodiments of the subject invention, the diameter of the tubular shaped body may be less than 8 inches.

In further embodiments of the subject invention, the length of the body may be at least 20 inches.

In additional embodiments of the subject invention, the length of the body may be at least 3 times the diameter.

In other embodiments of the subject invention, the length of the body may be substantially equal to the width of a bed size selected from the group consisting of twin size bed, full size bed, queen size bed and king size bed.

In additional embodiments of the subject invention, the insert may be a removable heating unit, additional cushioning, a vibration means, or a standard heating blanket.

In further embodiments of the subject invention, the cover may be a unitary cover; or a cover that comprises a first cover on the top of the pillow and a second cover on the bottom of the pillow.

In other embodiments of the subject invention, the power supply may have a cord attached thereto for selectively connecting to an electrical outlet or the power supply may be a battery.

In additional embodiments of the subject invention, the heating unit may be comprised of a plurality of heating coils within the body.

In other embodiments of the subject invention, the body may have a cross sectional shape selected from the group consisting of substantially circular, oval, square, triangular, pentagonal, octagonal, or hexagonal.

There have thus been broadly outlined important features of the invention in order that the detailed description thereof that follows may be better understood, and in order that the present contribution to the art may be better appreciated. There are additional features of the invention that will be described hereinafter and which will form the subject matter of the claims appended hereto. These together with other embodiments of the invention, and with various features of novelty which characterize the invention, are pointed out with particularity in the claims annexed to and formed as part of this disclosure.

For a conceptual understanding of the invention and its operational advantages, refer to the accompanying drawings and descriptive matter in which there are preferred embodiments of the invention illustrated. Other features and advantages of the present invention will become apparent from the following description of the preferred embodiment(s), taken in conjunction with the accompanying drawings, which by way of example; illustrate the principles of the invention.

### BRIEF DESCRIPTION OF THE DRAWINGS

Advantages of the present invention will be apparent from the following detailed description of exemplary embodiments thereof, which description should be considered in conjunction with the accompanying drawings:

FIG. 1 illustrates a top view of one embodiment of the pillow.

FIG. 2 illustrates a perspective top view of one embodiment of the pillow.

FIG. 3 illustrates a perspective top view of one embodiment of the pillow over the feet of a user lying in the supine position.

FIG. 4 illustrates a perspective top view of one embodiment of the pillow underneath the feet of a user lying in the supine position.

FIG. 5 illustrates a side view of one embodiment of the pillow over a shoulder of a user.

FIG. 6 illustrates a front view of one embodiment of the pillow around the neck of a user.

FIG. 7 illustrates a perspective top view of one embodiment of the pillow underneath the feet of two users lying in the supine position.

### DETAILED DESCRIPTION OF EMBODIMENTS

The following will describe, in detail, several embodiments of the present invention. These embodiments are provided by way of explanation only, and thus, should not unduly restrict the scope of the invention. In fact, those of ordinary skill in the art will appreciate upon reading the present specification and viewing the present drawings that the invention teaches many variations and modifications, and that numerous variations of the invention may be employed, used and made without departing from the scope and spirit of the invention.

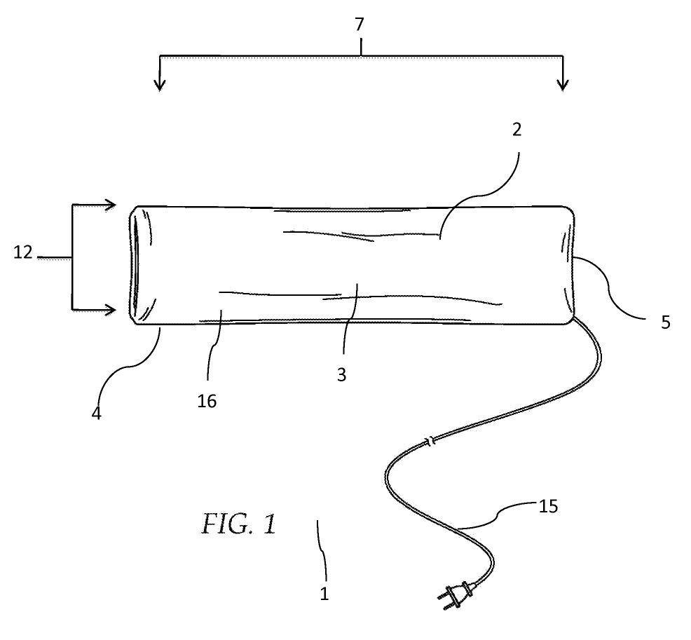

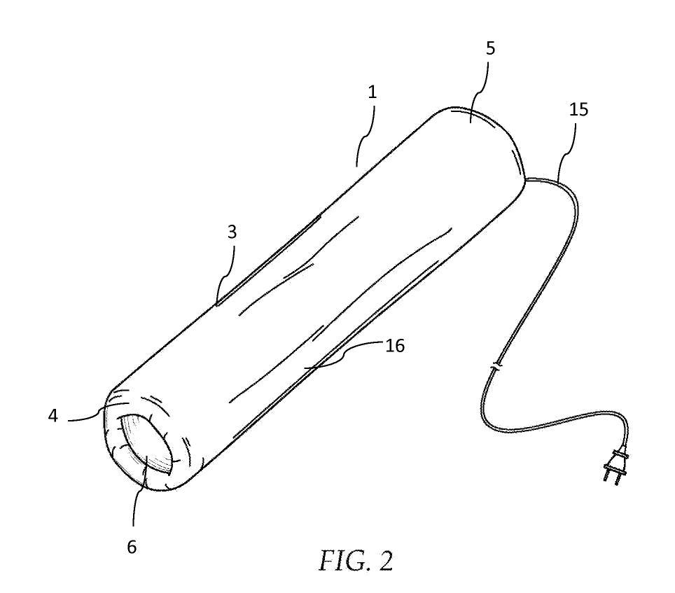

FIGS. 1-7 illustrate one embodiment of the pillow 1 of the subject invention. In this embodiment, the pillow 1 comprises a tubular-shaped body 2 composed of soft, flexible, resilient materials. These materials may include, but are not limited to foam, feathers, synthetic fills, down or other materials known to those skilled in the art.

Body 2 has a central portion 3, and two ends 4 and 5. Either end 4 or 5 may contain openings 6 to an internal channel (not shown) within body 2. This internal channel may slidably receive a heating unit, a vibrating unit, additional cushioning or a standard heating blanket. Thus, a user may easily rotate between additional cushioning, heat and vibration when using pillow 1.

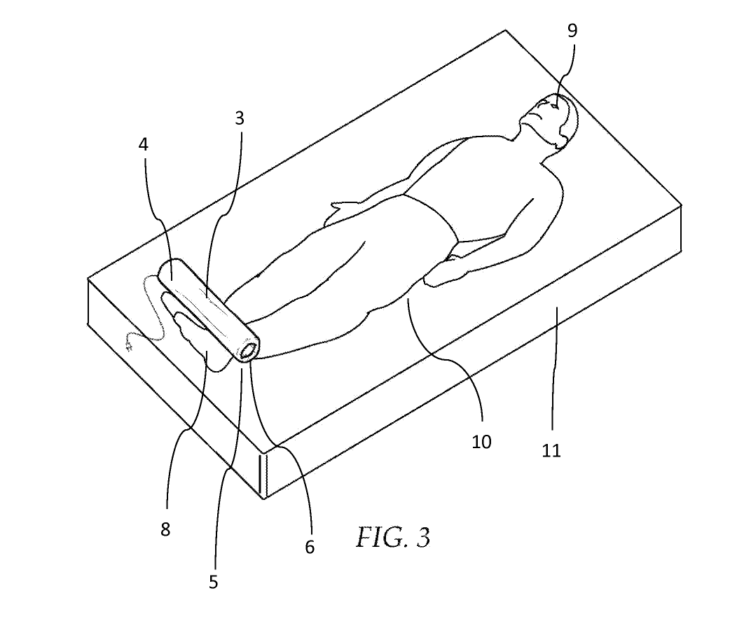

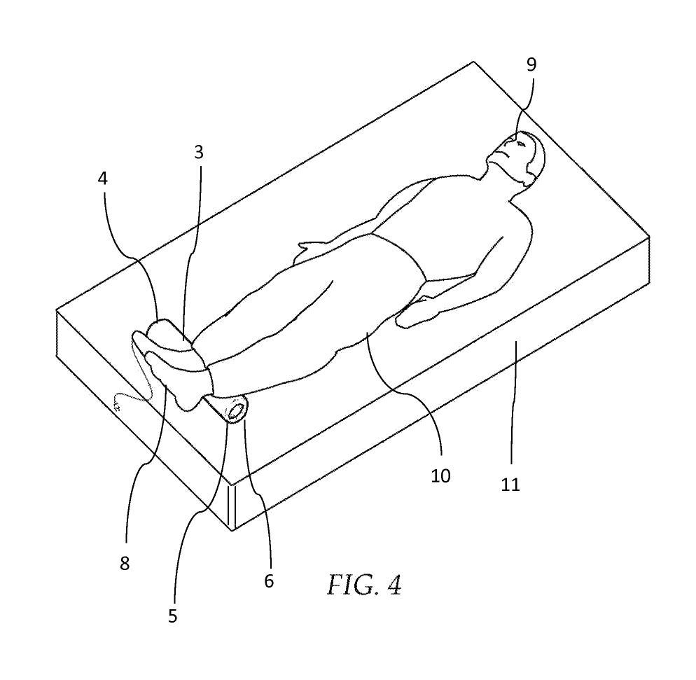

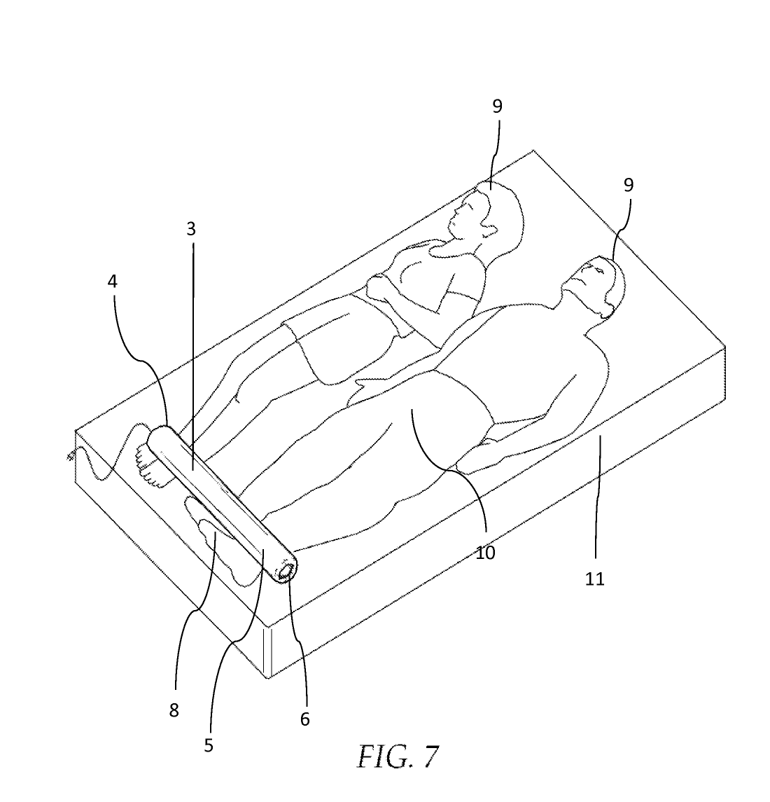

The length 7 of the tubular-shaped body 2 should be long enough to rest underneath or over both feet 8 of a user 9 lying in the supine position 10 (as shown in FIGS. 3 and 4) in a bed 11. In one embodiment of the subject invention, this length 7 of body 2 is long enough to substantially equal the width of a twin-size bed, full-size bed, queen size bed or a king size bed (not shown) to allow one or more users 9 lying supine in the bed 11 to rest the pillow 1 over or underneath their feet 8 (as shown in FIG. 7). The diameter 12 of the body 2 should be less than eight inches. In one embodiment, the length 7 of body 2 may be at least three times the size of diameter 12.

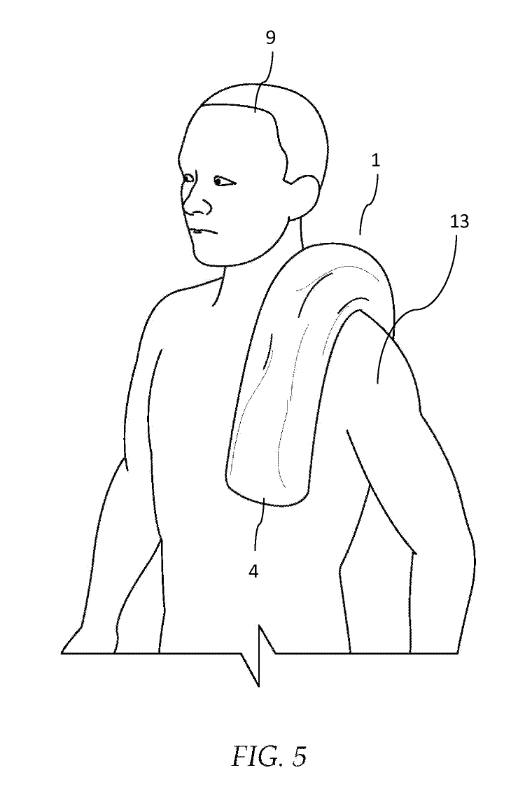

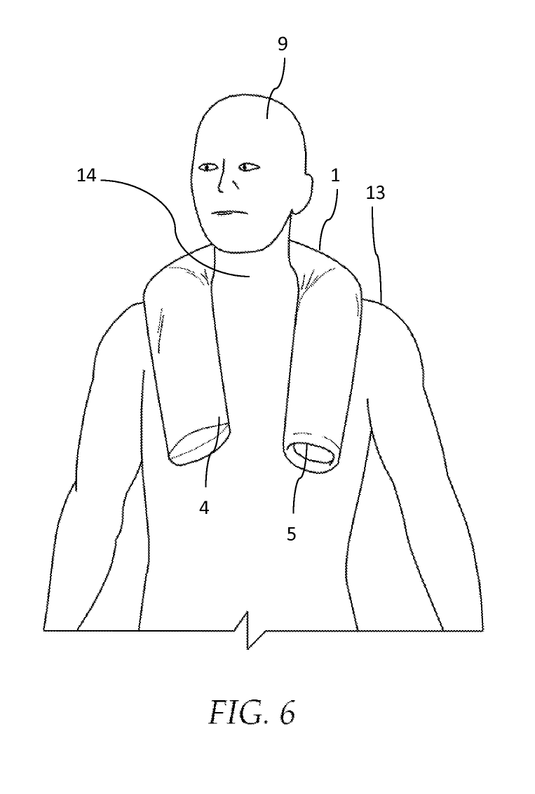

As shown in FIG. 5, the length 7 and diameter 12 of body 2 may be adapted to rest the pillow 1 over both sides of a shoulder 13 such that end 4 contacts the upper chest and end 5 contacts the upper back of user 9. As shown in FIG. 6, the length 7 and diameter 12 of body 2 may be adapted to rest the pillow 1 around the neck 14 of a user 9 such that ends 4 and 5 both contact the upper chest of a user 9.

In other embodiments (not shown), the pillow 1 has a size adapted for rest around a user's head, upper or lower back, or both legs.

The heating unit may be a plurality of heating coils permanent affixed within body 2. The heating or vibrating units of the subject invention may be operational coupled to a power supply that may have a cord 15 attached thereto for selectively connecting to an electrical outlet. In another embodiment of the subject invention, the power supply may be an internal battery.

In other embodiments of the subject invention, the pillow 1 may have a user-actuator operational coupled to the power supply and adapted to activate the heating unit.

The pillow 1 has a cover 16 that is configured for covering the external surface of the body 2. The cover 16 may also be configured for covering the internal surface of the internal channel. In further embodiments of the subject invention, the cover 16 may be a unitary cover, or two covers attached to one another.

The many aspects and benefits of the invention are apparent from the detailed description, and thus, it is intended for the following claims to cover such aspects and benefits of the invention, which fall within the scope, and spirit of the invention. In addition, because numerous modifications and variations will be obvious and readily occur to those skilled in the art, the claims should not be construed to limit the invention to the exact construction and operation illustrated and described herein. Accordingly, all suitable modifications and equivalents should be understood to fall within the scope of the invention as claimed herein.

What is claimed is:

1. (canceled)

2. A pillow comprising: a substantially tubular shaped body, wherein the body is composed of soft materials, further wherein the body comprises a first end, a second end and a central portion, wherein the central portion has a sufficient length such that the length is substantially equal to the width of a full size bed, and the body has sufficient flexibility for a user to wrap the central portion around a neck of a user such that the first end is adapted to contact an upper right chest of such a user and the second end is adapted to contact an upper left chest of such a user; a first opening on the first end; a second opening on the second end; an internal channel within the body; and a cover configured for covering the external surface of the body and the internal surface of the internal channel.

3. (canceled)

4. The pillow of claim 2, wherein the diameter of the body is less than 8 inches.

5. The pillow of claim 2, wherein the length of the body is at least 20 inches.

6. The pillow of claim 2, wherein the length of the body is at least 3 times the diameter.

7.-16. (canceled)

17. The pillow of claim 2, wherein the pillow has a cross sectional shape selected from the group consisting of substantially circular, oval, square, triangular, pentagonal, octagonal, and hexagonal.
